(19) United States
(12) Patent Application Publication
(10) Pub. No.: US 2011/0142775 A1
(43) Pub. Date: Jun. 16, 2011

(54) COSMETIC AND DERMATOLOGICAL FORMULATIONS WITH NATURAL PIGMENTS AND METHODS OF USE

(75) Inventors: Richard Hughes Kostick, Oakland, CA (US); Susie Pei Hwa Wang, Oakland, CA (US); James Pei Fung Wang, Oakland, CA (US)

(73) Assignee: VELVETEEN BUNNI LLC, Alameda, CA (US)

(21) Appl. No.: 13/032,845

(22) Filed: Feb. 23, 2011

Related U.S. Application Data
(63) Continuation of application No. 11/693,615, filed on Mar. 29, 2007, now abandoned.
Application No. 11/693,615 is a continuation-in-part of application No. 10/908,909, filed on May 31, 2005, now abandoned.

(51) Int. Cl. A61K 8/97; A61Q 19/00

(52) U.S. Cl. 424/64; 424/63

(57) ABSTRACT

Pigments from plant products can be combined with a cosmetic carrier formulation to create a cosmetic and/or dermatological product for transferring color to the skin of the person. A plant product extract or reformulation, such as a concentrate, flake or powder of the plant, can be combined with a cosmetic carrier formulation to create a cosmetic and/or dermatological product for transferring color to the skin of the person. In some embodiments, fruits, vegetables, seeds and legumes can be used to extract natural pigment or reformulate a plant product into a concentrate, flake or powder. Resultant cosmetic products can include lipstick, lip gloss, lip stain, lip liner, blush, face tint, cheek stain, cheek gel, cheek butter, eye shadow, eyebrow powder, eyeliner, mascara, foundation, sheer foundation, bronzer, facial illuminator, facial highlighter, face powder, lotion and tinted moisturizer.

### CROSS REFERENCE TO RELATED APPLICATIONS

This is a continuation-in-part of application Ser. No. 10/908,909, filed May 31, 2005, now pending.

### FIELD OF INVENTION

Cosmetic and dermatological formulations.

### BACKGROUND OF INVENTION

The use of cosmetics is widespread in modern society. Cosmetics typically are intended to provide an attractive appearance through the use of color, e.g., by highlighting certain features of the face and/or accentuating natural colors. Colored cosmetics are used, for example, to accentuate lines of separation (eye liners and lip liners), to provide sensuous color to portions of the skin (lipsticks and glosses) and to provide a “healthy glow” to the cheeks (blushes and rouges). Cosmetics may also be used to hide imperfections of the skin and to protect the skin, e.g., by blocking the skin from harmful ultraviolet light.

A variety of coloring agents are typically used to color cosmetics, including inorganic and organic synthetic dyes or pigments. Many cosmetic manufacturers use artificial or man-made pigments approved by the Food and Drug Administration designated as an FD&C “color” followed by a number. Examples of such pigments include FD&C Blue No. 1, FD&C Blue No. 2, FD&C Green No. 3, FD&C Red No. 3, FD&C Red No. 40, FD&C Yellow No. 5, FD&C Yellow No. 6, D&C Blue No. 4, D&C Blue No. 9, D&C Green No. 5, D&C Green No. 6, D&C Green No. 8, D&C Orange No. 4, D&C Orange No. 5, D&C Orange No. 10, D&C Orange No. 11, D&C Orange No. 17, FD&C Red No. 4, D&C Red No. 6, D&C Red No. 7, D&C Red No. 8, D&C Red No. 9, D&C Red No. 17, D&C Red No. 19, D&C Red No. 21, D&C Red No. 22, D&C Red No. 27, D&C Red No. 28, D&C Red No. 30, D&C Red No. 31, D&C Red No. 34, D&C Red No. 39, FD&C Red No. 40, D&C Violet No. 2, D&C Yellow No. 7, Ext. D&C Yellow No. 7, D&C Yellow No. 8, D&C Yellow No. 11, D&C Brown No. 1, Ext. D&C Violet No. 2, D&C Blue No. 6 and D&C Yellow No. 10.

Generally, to be useful as cosmetic colorants, soluble dyes must be converted into insoluble forms. Any soluble dye that is in solution can be referred to as a “solvated dye”. A solvated dye composed of a water-soluble dye in an aqueous solution can be referred to as an “aqueous dye”. There are a variety of methods currently used in the cosmetic industry to insolubilize soluble dyes. A common method used to insolubilize water-soluble dyes is called “laking”. “Lake” colorants are metallic complexes of organic coloring matter obtained by precipitating an organic dyestuff onto an inorganic substrate. See generally U.S. Pat. No. 3,873,687. Water-soluble dyes have also been insolubilized by salifying the dyes with copolymers. See generally U.S. Pat. No. 4,438,140.

Although FD&C colors are primarily used to formulate a cosmetic product, another class of pigments that are conventionally used are minerals, which are materials extracted from the earth, including, for example, iron oxides. Also used to formulate cosmetics are animal byproducts, such as blood and carmine.

### SUMMARY OF INVENTION

Pigments from plant products can be combined with a cosmetic carrier formulation to create a cosmetic and/or dermatological product for transferring color to the skin of the person. A plant product extract or reformulation, such as a concentrate, flake or powder of the plant, can be combined with a cosmetic carrier formulation to create a cosmetic and/or dermatological product for transferring color to the skin of the person. In some embodiments, fruits, vegetables, seeds and legumes can be used to extract natural pigment or reformulate a plant product into a concentrate, flake or powder. Resultant cosmetic products can include lipstick, lip gloss, lip stain, lip liner, blush, face tint, cheek stain, cheek gel, cheek butter, eye shadow, eyebrow powder, eyeliner, mascara, foundation, sheer foundation, bronzer, facial illuminator, facial highlighter, face powder, lotion and tinted moisturizer.

### DETAILED DESCRIPTION

According to embodiments of the invention, pigments extracted from fruits, vegetables, seeds, flowers and legumes can be used as a colorant in cosmetic and dermatological products. According to other embodiments, a reformulation product from fruits, vegetables, seeds, flowers and legumes, such as, but not limited to, a powder, flakes or concentrate, can be used as a colorant in cosmetic and dermatological products. The colorant is suitable for transfer to the skin when the cosmetic and/or dermatological product is applied thereon. In this manner, the pigment or reformulant is a “staining” colorant used to highlight and accentuate natural colors of the face. Such natural pigments can replace artificial, man-made pigments commonly used in cosmetic and dermatological products.

A “botanical product” is a constituent of a plant or tree. A botanical product can include, but is not limited to, stems, leaves, bark, fruit, skin of fruit, vegetables, flowers, seeds, legumes or any organic constituent of a plant or tree. According to some embodiments, a botanical product, or plant product (hereinafter used interchangeably), can be a fruit (including all constituents thereof, such as pulp and the skin of fruit) or a vegetable. Generally, fruits and vegetables are composed of a variety of compounds, including, water, carbohydrates, fats, lipids, proteins, amino acids, vitamins, minerals (e.g., phosphorous, potassium, calcium, magnesium and iron), organic acids, pectins, pectic enzymes and odorous compounds. Fruits and vegetables also contain pigments including, but not limited to, chlorophylls, anthoxanthins, betacyanins, carotenoids such as xanthophylls and carotenes, and flavonoids such as flavones and anthocyanidins (e.g., cyanidin, pelargonodin, delphinidin, malvidin, paenidin).

Anthocyanins, which are anthocyanidins with a sugar group, are water-soluble vacuolar flavonoid pigments that appear red to blue, according to pH. They are synthesized exclusively by organisms of the plant kingdom, and have been observed to occur in all tissues of higher plants, providing color in leaves, stems, roots, flowers, and fruits. Anthocyanin pigment is present in fruits such as, but not limited to, strawberries, pomegranates, cranberries, cherries and berries, and is responsible for their red color. Anthocyanins are a sub-category of flavonoids, and flavonoids are a sub-category of polyphenols. Lycopene is a bright red carotenoid pigment, a phytochemical found in tomatoes and other red fruits. Lycopene is a sub-category of tetraterpenes, and tetraterpenes are a sub-category of terpenes. Chlorophyll pigment is present in vegetables such as, but not limited to, green vegetables including beet greens, bok choy, collards, dandelion greens, kale, mustard greens and blue-green algae and is responsible for their green color. Carotenoid pigment, a carotene, is present in, for example, carrots and is responsible for their bright orange color. Anthoxanthin pigment is present in, for example, alfalfa flowers and red cabbage and is naturally a white color.

In some embodiments, the skin or pulp of fruit or vegetables can be processed to extract pigments such as anthocyanins and lycopenes. The resulting pigment extract can be in liquid or solid form. These various pigments in the form of extractions can be combined to produce numerous colors and used to formulate cosmetic and/or dermatological products according to embodiments of the invention. These plant products can be subjected to a process to isolate the pigments therefrom to be used to formulate a cosmetic or dermatological product according to some embodiments of the invention.

### EXAMPLE 1

In one example, a plant sample, such as the seed, skin or pulp of fruit can be subjected to a “quick freeze” in liquid nitrogen and then subsequently freeze-dried. The sample can thereafter be subjected to at least 3 washes with hexane. The hexane washes are used to remove lipid from the plant sample (if the sample contains lipids). The hexane should preferably be optima grade hexane (available from Fischer Scientific, Waltham, Mass.) or equivalent. Polyphenols in the sample can then be extracted using a solvent system such as methanol/water (75:25 v/v). The methanol should preferably be high-performance liquid chromatography (HPLC) grade methanol (available from Sigma-Aldrich, St. Louis, Mo.) and the water should preferably be purified to about 18.2 megaohms per centimeter (MΩ/cm). In some embodiments, the extraction step can be performed multiple times. The extract can then be filtered through a 0.45 micron polytetrafluoroethylene (PTFE) membrane, or other size depending on the sample size.

### EXAMPLE 2

In another example, a plant sample such as the seed, skin or pulp of fruit can be subjected to a “quick freeze” in liquid nitrogen and then subsequently freeze-dried. The sample can be ground down using a mortar and pestle in optima grade hexane. The sample can then be washed with hexane and centrifuged for about 10 minutes at a speed of about 4000 to 5000 rotations per minute. This removes lipids from the sample (if the sample contains lipids). This can be done for at least 3 cycles, letting the particulates settle between each wash/centrifugation. Thereafter, the remaining precipitate is extracted using a solvent system such as methanol/water (75:25 v/v) for about 24 hours at approximately 5 degrees Celsius.

In other embodiments, fruit or vegetables can be reformulated to obtain a powder, flake or concentrate (collectively, plant product reformulation) of the fruit or vegetable, which also can include the pigments described previously. This plant product reformulation, or reformulant, can also be used to formulate cosmetic and/or dermatological products according to embodiments of the invention. In the context of this Application, “reformulate” means to alter a plant product from its natural state to an altered state such as a concentrate, flake or powder.

In one example, to reformulate a vegetable into a powder, the following alternative processes can be used: (a) dry vegetable down to a final water content below 4%, followed by grinding, followed by sieving; or (b) boil vegetable, followed by sieving into a puree, followed by drying on a heated surface (preferably under vacuum) or spraying in hot air. The drying processes can encompass one of the following: (i) dryers with plates under vacuum are equipped with plates heated with hot water; stainless steel plates containing purée to be dried are placed on them; process conditions are at low residual pressure (about 10 to 20 mm Hg) and a product temperature of between 50° C. to 70° C.; (ii) drum dryers having one or two drums heated with hot water or steam as heating elements; feeding is continuous between the two drums which can rotate in reverse direction (about 2-6 rotations per minute) and the distance of which is adjustable and determines the thickness of layer to be dried; the product can be dried and removed by mechanical means during rotation; or (iii) drying installations by spraying in hot air; the product is introduced in equipment and sprayed by a special device in hot air; drying is instantaneous ( 1/50 s) and therefore can be carried out in a range of between 130° C. to 150° C.

In another example, to reformulate a fruit such as a tomato into a concentrate, the following process can be used: pre-wash, wash and sort tomatoes, followed by tomato crushing and seed separation with a centrifugal separator. The resulting tomato pulp is pre-heated at 55° C. to 60° C. and then passed to an equipment group for sieving: pulper, refiner and super-refiner with sieves of 1.5 mm, 0.8 mm and 0.4 to 0.5 mm, respectively. The resulting tomato juice is concentrated by vacuum evaporation. For example, the tomato juice can be subjected to a 3-step evaporation method: (i) pasteurize juice at between 85° C. to 90° C. for 15 minutes at 330 mm Hg; (ii) heat resulting concentrate at between 42° C. to 46° C. at between 680 mm Hg to 700 mm Hg; and heat concentrate again at between 42° C. to 46° C. at between 680 mm Hg to 700 mm Hg. Force resulting concentrate through tubular pasteurizer at between 90° C. and 92° C.

Alternatively, freeze drying or dehydration of a fruit, vegetable, fruit skin or vegetable skin can result in fruit or vegetable powder or flakes that can be used to formulate cosmetic and/or dermatological products according to embodiments of the invention. “Freeze drying” is the process of freezing a material and then reducing the surrounding pressure and adding enough heat to allow the frozen water in the material to sublime directly from the solid phase to gas. “Dehydration” is the process of removing water from an object. Dehydration of fruits and methods can be performed by known methods. In some embodiments, the dehydrated (or freeze dried) plant product can be micronized to create the powder. “Micronized” means to reduce to particles that are only a few microns in diameter. In some embodiments, the concentrate, powder or flakes can be the direct source of color transfer from the cosmetic and/or dermatological product, eliminating the need for color additives such as FD&C colors and/or Lake minerals. However, those skilled in the art will appreciate that the use of FD&C colors and/or Lake minerals in embodiments of the cosmetic and/or dermatological formulations of the invention may also be included.

In some embodiments, the botanical product can be a flower. Examples of flowers which can be used according to embodiments of the invention include, but are not limited to, roses (all varietals), violets, lavender, tulips, chrysanthemums, petunias, daffodils, poppies, orchids, begonia, lilies, pansies, daisies, hibiscus, carnations, and blossoms including cherry blossoms and jacqueranda blossoms. Such flowers may include at least one pigment such as anthocyanins, flavones, carotenes and chlorophyll. Pigment extraction can be performed by the processes described previously.

In some embodiments, the botanical product can be a seed, such as a coffee seed (commonly referred to as a coffee bean) or a cocoa seed (commonly referred to as a cocoa bean). In unfermented cocoa beans, pigment cells make up about 11-13% of the tissue. The pigments in unroasted cocoa seeds include both anthocyanins and polyphenols. During fermentation, polyphenols and anthocyanins undergo different chemical reactions that lead to the synthesis of flofabens, which contribute to the characteristic brown color of roasted cocoa seeds. Additionally, oxidation and polymerization of polyphenols during the roasting process of cocoa seeds leads to their characteristic brown color. Similarly, coffee seeds contain brown pigments. A pigment extract derived from cocoa or coffee can be in liquid or solid form and can be used to formulate a cosmetic and/or dermatological product according to embodiments of the invention. Alternatively, concentrate, flakes or micronized powder from the cocoa or coffee bean can be the direct source of color transfer from the cosmetic and/or dermatological product, eliminating the need for color additives such as FD&C colors and/or Lake minerals.

In some embodiments, the botanical product can be a bean, such as a black bean or a red bean. Pigments can be extracted from the hull, or coat, of red and black beans. The pigments cyanidin 3-O-β-D-glucoside (RPO) and pelargonidin 3-O-β-D-glucoside (RP1) can be extracted from the red bean, while the pigment deiphinidin 3-O-β-D-glucoside (BP1) can be extracted from the black bean. RPO is a flavonoid, more specifically an anthocyanin, and responsible for the blue, purple and red color of many plant tissues. RP1 is also a flavonoid, more specifically an anthocyanin, and responsible for the orange, pink, red and magenta color of many plant tissues. Similarly, BP1 is a flavonoid responsible for the purple color of, for example, black bean hull. The resulting pigment extract can be in liquid or solid form.

In sum, at least one of the following plant products or constituent thereof (concentrate, flake, powder) can be used to formulate a cosmetic or dermatological product according to embodiments of the invention: apricot, peach, nectarine, cherry, plum, raspberries, strawberry, blackberry and derivative and hybrid species thereof, (e.g., dewberry, boysenberry, olallieberry and loganberry, wineberry, bearberry, bilberry or whortleberry), blueberry, cranberry, lingonberry, currant (including red, black, and white types), elderberry, grape, mulberry, persimmon, papaya, butternut squash, pumpkin, acorn squash, summer squash, rhubarb, jujube, black mulberry, olive, pomegranate, guava, kumquat, passion fruit, guava, açai, durian, mango, mangosteen, papaya, pineapple, broccoli, brussel sprouts, cabbage, cauliflower, kale, rapini, arugula, pumpkin, tomato, spinach, eggplant, corn, beets, peppers, black beans, azuki beans, carrots, as well as flowers, tea leaves and seaweeds.

The pigment extracts and/or plant product reformulations according to embodiments of the invention can be combined with, for example, a cosmetic carrier. A cosmetic carrier is a formulation that can be safely applied to the skin of a person and is principally used for beautifying effects and ease of application. The cosmetic carrier may be composed of a wide variety of ingredients that are conventionally used in cosmetics, e.g., waxes, mineral oils, fatty alcohols, glycerine, and sunscreens. In one embodiment, the cosmetic carrier may be a combination of rhea butter, cocoa butter, grapeseed oil, honey beeswax, vitamin C, vitamin E and a natural flavor. When the pigment extract or plant product reformulation (e.g., powder, flakes, concentrate) are combined with a cosmetic carrier, the resulting formulation can be used to make a variety of cosmetic and/or dermatological products, such as, but not limited to, lipstick, lip gloss, lip stain, lip liner, blush, face tint, cheek stain, cheek gel, cheek butter, eye shadow, eyebrow powder, eyeliner, mascara, foundation, sheer foundation, bronzer, facial illuminator, facial highlighter, face powder, lotion and tinted moisturizer.

According to some embodiments, pigment extracts or plant product reformulations (e.g., powder, flakes, concentrate) in a range of between about 0.01% by mass (weight percent) and about 99.9% can be combined with a cosmetic carrier. For example, in one embodiment, a cosmetic/dermatological formulation includes about 83% vitamin E, about 10% pigment extract or plant product reformulation from pomegranate fruit, about 5% pigment extract or plant product reformulation from cherry fruit and about 2% pigment extract or plant product reformulation from strawberry fruit, wherein the pigments are adapted to stain and/or color the skin of a person when applied. In some embodiments, the formulation can alternatively include at least one of grapeseed oil, honey, shea butter, honey beeswax, vitamin C, natural fruit flavor, chili pepper or red wine. In one embodiment, the formulation is a lip gloss.

In another embodiment, a face tint (commonly referred to as foundation) can include about 99.9% shea butter and about 0.01% cocoa bean pigment, wherein the pigment is adapted to stain and/or color the skin of a person when applied. In another embodiment, a blush can include 100% reformulated micronized fruit powder from raspberry fruit, wherein the pigment is adapted to stain and/or color the skin of a person when applied. In another embodiment, a formulation includes In one embodiment, the formulation is a face tint which can be applied to the lips, checks or eyelids.

In addition to the non-plant product ingredients listed above, cosmetic/dermatological products formulated according to embodiments of the invention can include a number of fillers and preservatives which are listed in the examples that follow. Thus, the following are examples of cosmetic/dermatological products formulated in accordance with embodiments of the inventions (all percentages provided are by weight unless otherwise noted):

A lipstick formula (pink shade) can include 4.72% polyethylene, 5.67% White SP-1020, 8.06% diisostearyl malate, 12.6% dipentaerythrityl hexahyrdroxystearate/hexasterate/hexarosinate, 9.0% petroatum, 0.6% grapeseed oil, 16.41% diisospropyl dimer dilinoleate, 10.0% isononyl isononanoate, titanium dioxide, stearic acid and aluminum hyroxide, 8.0% diisospropyl dimer dilinoleate, 5.44% fruit pigment extract and/or fruit product reformulation containing anthocyanins, 2.0% mica and isopropyl titanium triisostearate, 3.0% mica and titanium dioxide, 8.0% diisospropyl dimer dilinoleate, 5.0% silica and 1.5% peppermint essential oil.

A lipstick formula (red orange shade) can include 4.72% polyethylene, 5.67% White SP-1020, 8.06% diisostearyl malate, 12.6% dipentaerythrityl hexahyrdroxystearate/hexasterate/hexarosinate, 9.0% petroatum, 0.6% grapeseed oil, 16.41% diisospropyl dimer dilinoleate, 10.0% isononyl isononanoate, titanium dioxide, stearic acid and aluminum hyroxide, 8.0% diisospropyl dimer dilinoleate, 5.44% pigment extract and/or fruit product reformulation containing carotenoids and/or tetraterpenes (lycopene), 2.0% mica and isopropyl titanium triisostearate, 3.0% mica and titanium dioxide, 8.0% diisospropyl dimer dilinoleate, 5.0% silica and 1.5% peppermint essential oil.

A mascara formula (purple shade) can include 36.91% isododecano, 14.29% hydrogenated polycyclopetadiene and isododecano, 2.0% lithium magnesium silicate, 1.0% SD Alcohol—39C, 13.25% pigment extract and/or fruit product reformulation containing anthocyanins, 0.25% sodium chloride, 13.5% deionized water, 0.45% PHENONIP®, 5.0% C18-36% triglycerides, 6.0% beeswax, 3.0% carnauba wax, 3.0% polyethylene and 0.35% panthenol.

A powdered blush formula (red shade) can include 72.85% kaolin powder, 3.1% pigment extract and/or fruit product reformulation containing lycopene, 1.75% pigment extract and/or fruit product reformulation containing anthocyanins, 9.0% mica and titanium dioxide, 0.30% vitamin E and 13.0% dimethicone.

An eye shadow formula (blue eye shadow) can include 69.70% sericite and triethoxycaprylysilane, 7.0% pigment extract and/or fruit product reformulation containing anthocyanins, 10.3% mica and titanium dioxide, 13.0% dimethicone and triemethylsiolxysilicate.

A powdered foundation can include 42.0% lecithin, 40.0% kaolin powder, 3.26% titanium dioxide and isoproryl titanium triisosterate, 0.88% pigment extract and/or fruit product reformulation containing carotenoid, 5.76% silica and 8.0% pentaerythritol tetraoctanoate.

A cheek tint (e.g., shimmery cocoa berry) can include 32.0% cocoa butter, 25.0% shea butter, 13.0% grapeseed oil, 10.0% honey beeswax, 5.0% vitamin E, 4.0% cocoa bean fruit pigment, 2.5% raspberry fruit pigment, 2.0% blackberry fruit pigment, 2.0% vitamin C, 1.5% natural strawberry flavor, 1.0% pomegranate fruit pigment, 1.0% red wine pigment and 1.0% strawberry fruit pigment.

A cheek tint (e.g., shimmery strawberry) can include 32.0% cocoa butter, 25.0% shea butter, 13.0% grapeseed oil, 10.0% honey beeswax, 5.0% vitamin E, 2.8% cranberry fruit pigment, 2.0% cherry fruit pigment, 2.0% vitamin C and 1.5% natural strawberry flavor.

A cheek tint (e.g., sugar plum sheer) can include 32.0% cocoa butter, 25.0% shea butter, 10.0% honey beeswax, 5.0% plum fruit pigment, 5.0% grapeseed oil, 5.0% vitamin E, 4.0% cranberry fruit pigment, 3.0% pomegranate fruit pigment, 3.0% blackberry fruit pigment, 2.0% raspberry fruit pigment, 2.0% red wine fruit pigment, 2.0% vitamin C, 1.5% natural strawberry flavor and 0.5% strawberry fruit pigment.

When applied, the fruit pigment formulated within the cosmetic or dermatological product can stain the skin immediately on application. In some embodiments, there is a time interval between the time the cosmetic and/or dermatological product is applied to the skin and the time that a pigment in the product stains the skin. A representative time interval from application to staining can be up to about 5 minutes, and, in one embodiment, 30 seconds to one minute. In any case, the stain is temporary, not permanent.

In the foregoing specification, specific embodiments have been described. It will, however, be evident that various modifications and changes can be made thereto without departing from the broader spirit and scope of the appended claims. The specification and drawings are, accordingly, to be regarded in an illustrative rather than a restrictive sense.

What is claimed is:

1. A method of coloring the skin of a person, comprising:
applying an effective amount of a cosmetic or dermatological formulation consisting of one of (i) a reformulated product of a plant product or (ii) a pigment extract from a plant product and a cosmetic carrier having admixed therein the reformulated product.

2. The method of claim 1, wherein the reformulated product or pigment extract is one of a concentrate, a powder or a flake of the plant product, the plant product selected from the group consisting of a fruit, a vegetable, a legume, a seed, a flower, a leaf and a combination thereof.

3. The method of claim 2, wherein the fruit, the vegetable, the flower, the leaf or the seed is at least one of apricot, peach, nectarine, cherry, plum, raspberry, strawberry, blackberry and derivative and hybrid species thereof, blueberry, cranberry, lingonberry, currant, elderberry, grape, mulberry, persimmon, papaya, butternut squash, pumpkin, acorn squash, summer squash, rhubarb, jujube, black mulberry, olive, pomegranate, guava, kumquat, passion fruit, guava, açai, durian, mango, mangosteen, papaya, pineapple, broccoli, brussel sprouts, cabbage, cauliflower, kale, rapini, arugula, pumpkin, tomato, spinach, eggplant, corn, beets, peppers, carrots, black beans, azuki beans, coffee seed, cocoa seed, tea and a flower wherein the flower is selected from the group consisting of roses, violets, lavender flowers, tulips and chrysanthemums, and optionally includes one of chili powder, seaweed or red wine.

4. The method of claim 3, wherein the cosmetic carrier further comprises at least one of shea butter, cocoa butter, grapeseed oil, peppermint essential oil, honey beeswax, carnauba wax, kaolin powder, sericite, vitamin C, vitamin E or a natural flavor.

5. The method of claim 4, wherein the formulation comprises one of a lipstick, a lip gloss, a lip stain, a lip liner, a blush, a face tint, a cheek stain, a cheek gel, a cheek butter, an eye shadow, an eyebrow powder, an eyeliner, a mascara, a foundation, a sheer foundation, a bronzer, a facial illuminator, a facial highlighter, a face powder, a lotion and a tinted moisturizer wherein the pigment is present in an amount to stain a portion of skin of a person when the cosmetic product is applied thereon.

6. The method of claim 1, wherein the reformulated product or pigment extract is non-permanent.

7. The method of claim 6, wherein the reformulated product or pigment extract stains the skin of a person immediately.

8. The method of claim 6, wherein there is a time interval between application of the formulation to the skin of a person and the time the pigment stains the skin of a person.

9. The method of claim 5, wherein the cosmetic formulation is a lip gloss having at least 83% vitamin E.

10. The method of claim 5, wherein the cosmetic formulation is a face tint having at least 32% cocoa butter and suitable for tinting cheeks, lips or eyelids.

11. A method of coloring the skin of a person, comprising:
applying an effective amount of a cosmetic or dermatological formulation consisting of a reformulated product of a plant product wherein the reformulated product is one of a powder, a micronized powder or a flake of the plant product, the plant product selected from the group consisting of a fruit, a vegetable and a combination thereof.

12. The method of claim 11, wherein the fruit, the vegetable, the flower, the leaf or the seed is at least one of apricot, peach, nectarine, cherry, plum, raspberry, strawberry, blackberry and derivative and hybrid species thereof, blueberry, cranberry, lingonberry, currant, elderberry, grape, mulberry, persimmon, papaya, butternut squash, pumpkin, acorn squash, summer squash, rhubarb, jujube, black mulberry, olive, pomegranate, guava, kumquat, passion fruit, guava, açai, durian, mango, mangosteen, papaya, pineapple, broccoli, brussel sprouts, cabbage, cauliflower, kale, rapini, arugula, pumpkin, tomato, spinach, eggplant, corn, beets, peppers, carrots, black beans, azuki beans, coffee seed, cocoa seed, tea and a flower wherein the flower is selected from the group consisting of roses, violets, lavender flowers, tulips and chrysanthemums, and optionally includes one of chili powder, seaweed or red wine.

13. The method of claim 12, wherein the formulation comprises one of a lipstick, a lip gloss, a lip stain, a lip liner, a blush, a face tint, a cheek stain, a cheek gel, a cheek butter, an eye shadow, an eyebrow powder, an eyeliner, a mascara, a foundation, a sheer foundation, a bronzer, a facial illuminator, a facial highlighter, a face powder, a lotion and a tinted moisturizer wherein the pigment is present in an amount to stain a portion of skin of a person when the cosmetic product is applied thereon.

14. The method of claim 11, wherein the formulation is a blush consisting of 100% of the reformulated product of a plant product, wherein the reformulated plant product is micronized raspberry powder.
